(19) United States
(12) Patent Application Publication
(10) Pub. No.: US 2026/0063314 A1
(43) Pub. Date: Mar. 5, 2026

(54) SYSTEM FOR RENOVATING FACILITIES TO PROVIDE A DISINFECTED AIR FLOW

(71) Applicant: Madhavan Pisharodi, Brownsville, TX (US)

(72) Inventor: Madhavan Pisharodi, Brownsville, TX (US)

(21) Appl. No.: 19/381,987

(22) Filed: Nov. 6, 2025

Related U.S. Application Data
(63) Continuation-in-part of application No. 18/896,765, filed on Sep. 25, 2024, now Pat. No. 12,465,537.
Application No. 18/896,765 is a continuation-in-part of application No. 18/328,463, filed on Jun. 2, 2023, now Pat. No. 12,194,206.
(60) Provisional application No. 63/586,322, filed on Sep. 28, 2023.

(51) Int. Cl. F24F 7/003; A61L 9/20; F24F 7/06; F24F 8/10; F24F 8/22

(52) U.S. Cl. CPC F24F 7/003; A61L 9/20; F24F 7/065; F24F 8/10; F24F 8/22; A61L 2209/12; A61L 2209/14; A61L 2209/16

(57) ABSTRACT

An air flow delivery system designed to more evenly deliver conditioned air to an enclosure. The system includes an air supplier attached to the attached air supply duct. The air supplier extends vertically toward the floor of the enclosure and contains an air supply vent that releases the conditioned air close to the floor of the enclosure, thereby increasing the circulation of the air throughout the entire enclosure. When the enclosure houses infectious disease patients, the air flow delivery system further includes a disinfection unit, so that the circulated conditioned air is disinfected to prevent the spread of the disease. Another embodiment includes: (a) an air intake pipe having a first bidirectional valve therein, wherein when the bidirectional valve is in an open state, ambient air enters the enclosure; (b) an air handler, the air handler coupled to a disinfection unit; (c) an air supply duct attached to the disinfection unit, wherein the air supply duct delivers conditioned and disinfected air from the air handler to one or more air supply vents positioned at or near a bottom surface of the enclosure; and (d) an air an air return duct having a first and a second bidirectional valve therein, wherein when the first bidirectional valve is in an open state, ambient air enters the air flow delivery system, and wherein when the bidirectional valve is in a closed state, ambient air is prevented from entering the air flow delivery system.

### CROSS REFERENCE TO RELATED APPLICATIONS

This application is a non-provisional application that claims priority to and is a continuation in part of U.S. Ser. No.: Ser. No. 18/896,765 filed on Sep. 25, 2024, which claims priority to U.S. Provisional Patent Application Ser. No. 63/586,322 filed on Sep. 28, 2023 and also claims priority to and is a continuation in part of U.S. Ser. No.: Ser. No. 18/328,463 filed on Jun. 2, 2023. The entire disclosures of these patent applications are part of the disclosure of the present application and are hereby incorporated by reference in their entireties.

### FIELD OF THE INVENTION

Embodiments of the invention relate generally to a system for renovating enclosures to provide a conditioned airflow more evenly throughout such enclosures.

### BACKGROUND

History has not been kind to individuals with infectious diseases or suspected of having infectious diseases. The general perception is that infections are self-inflicted and that in the interests of society at large, it is more important to isolate these individuals to protect others from being infected. Too often this allowed society to treat individuals with infections with disrespect and cruelty. Such thinking allowed society to segregate leper colonies from the rest of the population in the Middle Ages. Similar measures were used throughout the centuries for individuals suffering from deadly or disfiguring diseases that were contagious or merely suspected to be so. More recently, scientists have labeled this segregation “source-prevention”. Source prevention does not take into account the safety of the individuals who are deemed to be the source of the infection. For instance, if a patient infected with the SARS-CoV-2 virus is forced to wear a face mask, it could have deadly consequences because of the possibility of the patient re-inhaling the virus trapped in their mask. However, this risk is considered irrelevant or minimal since it prevents other individuals from being exposed to the virus. As such, there is an unmet need to effectively treat people with serious infections, especially the ones that are contagious, while preserving their dignity and ensuring that they are not re-infected.

Heating, ventilating and air conditioning (HVAC) systems are installed in confined spaces in order to heat/cool those confined spaces. A conventional central or split HVAC/air conditioning system 100 for a residential or commercial space, is shown in FIG. 1. Air that gets into the room is generally recirculated by the HVAC system. In a conventional airflow system, a supply vent is positioned on or substantially near the ceiling to deliver hot/cool air. The air is then circulated in the room. The circulated air/used air leaves the room through return vents which are also located on or substantially near the ceiling. Hot air rises while cold air sinks. The hot air in the room tries to go up towards the ceiling. Because of this conventional arrangement, air circulation is naturally more powerful close to the ceiling and gets weaker and cooler as it goes down towards the floor (as shown in FIG. 1). This leaves cold, dry, stagnant air in the lower part of the room where people usually move around. Only a portion of circulated/used air leaves the room through return vents located on the ceiling leaving the cold, dry air in the lowermost part of the room stagnant resulting in an ineffective air conditioning system. This air can go round and round in the room without ever getting recycled (as shown in FIG. 1). Unfortunately, cold, dry, stagnant air is ideal for airborne pathogens such as viruses (e.g. coronavirus), bacteria, and the like to remain infective.

Conventional central air conditioning systems include an air handler (usually located in an attic or basement of a residential space) and a condenser (usually located outside the residential space). Air handlers are devices that circulate conditioned air - that is, warmed or cooled air-throughout the space being air conditioned. The system 100, shown in FIG. 1, includes a supply vent and a return vent both positioned on or substantially near the ceiling to deliver hot/cold air to the space and to discharge the “used air” out of the space. Hot air that is circulated normally rises to the ceiling which causes the cold air to sink to the lower part of the space causing it to stagnate in the lower part of the room. In cold climates when a heater is turned on, the hot air coming through the supply vent traverses only close to the ceiling. Thus, in conventional air conditioning systems, more time and energy is required to heat the space because of the resistance for the hot air to go down towards the floor, leaving cold stagnant, dry air in the lower regions of the room. This is an avoidable waste of energy. Therefore, there is a need for improved air conditioning systems that deliver conditioned air throughout the room or enclosure.

In addition, changes in the habitats of non-domesticated animals are driving an increasing number of species out of their natural habitats and into interaction of humans, thereby increasing a rise in unknown diseases into the human population (e.g., the corona virus and the ebola virus). As epidemics spread, there is often a need to convert simple clinics into intensive care units with effective disease control. However, the resources for such renovations particularly in isolated areas of the world are simply not available. Thus, there is an ongoing need for a simple and inexpensive system for renovating existing structures and enclosures to be able to provide conditioned and/or disinfected conditioned air to the interior of existing enclosures.

### SUMMARY OF THE DISCLOSURE

Embodiments of the invention relate generally to a system for renovating facilities to provide conditioned air to existing enclosures.

One objective of the invention relates to an air flow delivery system for delivering conditioned air to an enclosure, comprising: (a) an air handler coupled at a first end to an air supply duct and at second end to an air return duct; (b) an air supply vent attached to the air supply duct, wherein the air supply vent is in or near the ceiling of the enclosure; (c) an air return vent attached to the air return duct, wherein the air return vent is in or near the ceiling of the enclosure; (d) an air supplier connected at a first end to the air supply vent, wherein the air supplier projects downward from the air supply vent into the enclosure near a floor of the enclosure, and wherein an air supply vent opening providing an air passage from the interior of the air supplier to the enclosure.

The air flow delivery system described above may be adapted to deliver disinfected conditioned air to an enclosure by installing a disinfection unit within the air supply line between the air supply vent and the air supply vent opening that provides an air passage from the interior of the air supply line to the enclosure.

Furthermore, the air flow delivery system described above may be adapted to have multiple air supply lines connected to the air vent is order to provide multiple downwardly projecting air supply lines, wherein each air supply line has an air supply line opening, thereby allowing multiple sites of entry for the disinfected conditioned air within the enclosure.

Another objective of the present invention is an air flow delivery system for delivering conditioned air to an enclosure, comprising: (a) an air intake pipe having a bidirectional intake valve therein, wherein when the bidirectional intake valve is in an open state, ambient air enters the air flow delivery system, and wherein when the bidirectional intake valve is in a closed state, ambient air is prevented from entering the air flow delivery system; (b) an air return duct having a first and a second bidirectional valve therein, wherein when the first bidirectional valve is in an open state and the second bidirectional valve is in a closed state ambient air enters the air flow delivery system, and wherein when the first bidirectional valve is in a closed state and the second bidirectional valve is in an open state, ambient air is prevented from entering the air flow delivery system; (c) an air handler coupled to the air intake pipe below the bidirectional intake valve in the air intake pipe, the air supply duct, and the air return duct; (d) an air supply duct connected to a second end of the air handler and extending into an interior of the enclosure, wherein the air supply duct delivers conditioned air from the air handler to one or more air supply vents positioned within the enclosure, wherein the air supply duct extends vertically from a ceiling of the enclosure toward a floor of the enclosure; and (e) one or more air return vents connected to a return duct for routing used air from the enclosure.

This system may include a disinfection unit in communication with the air handler and the air supply duct.

### BRIEF DESCRIPTION OF THE DRAWINGS

FIG. 1 illustrates a typical airflow pattern in a confined space having supply and return vents on the ceiling.

FIGS. 2A-2B illustrates a modified air delivery system for an enclosure according to an embodiment.

FIG. 2C illustrates a modified air delivery system according to an embodiment.

FIGS. 3A-3B illustrates a modified air delivery system with UVC disinfected air according to another embodiment.

FIG. 4A illustrates an air disinfection unit according to an embodiment.

FIG. 4B illustrates an air disinfection unit according to another embodiment.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

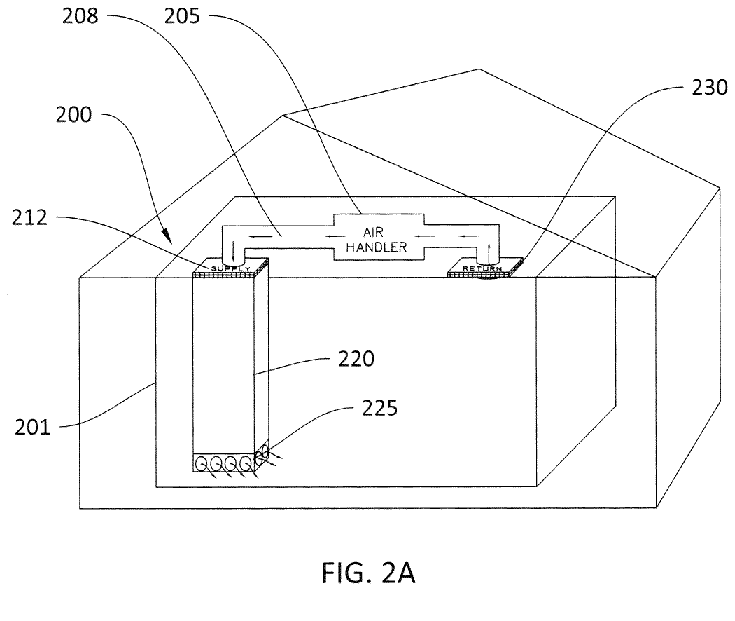

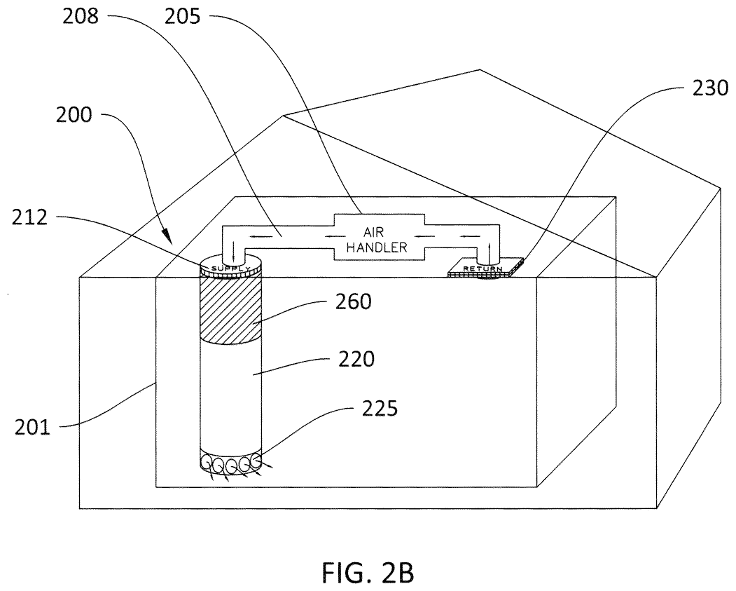

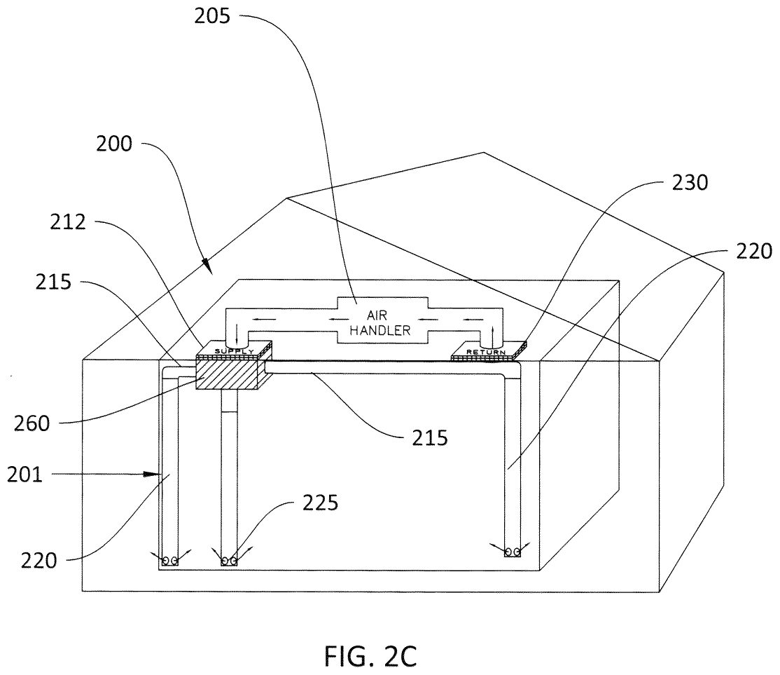

According to an embodiment, a modified air delivery system 200 for routing airflow within a confined space 201 is illustrated in FIGS. 2A and 2B. As shown, the system 200 is used to deliver fresh, chilled, or hot air to the enclosure 201. As used hereinafter, the term “enclosure” can include any confined space, such as, a pavilion, a building, a room, a nursing home, an airplane, a vehicle, a train, a ship, a critical care unit in a hospital, and a free-standing, mobile enclosure.

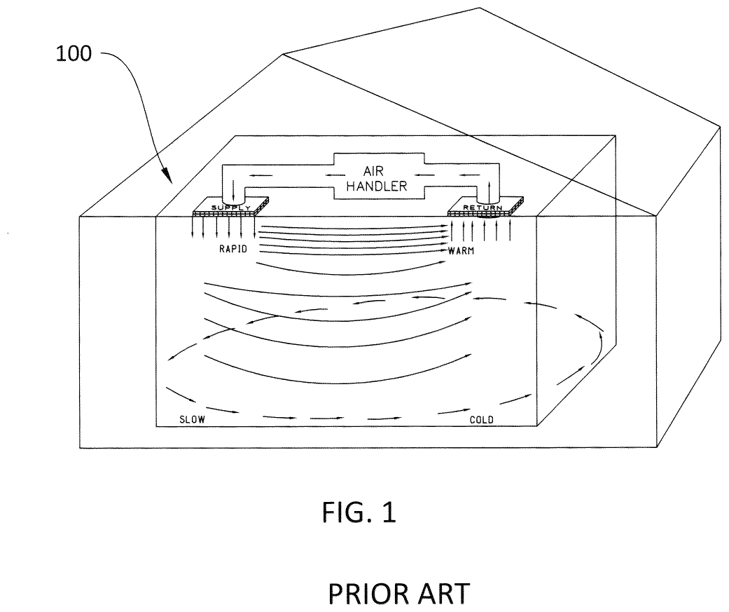

Similar to the conventional air conditioning system 100, the air conditioning system 200 includes an air handler 205 (usually located in an attic or basement of a residential space) and a condenser (usually located outside the residential space). Air handlers are devices that circulate conditioned air—that is, warmed or cooled air—throughout the space being air conditioned. The system 200, shown in FIG. 2A, includes a supply vent 212 and a return vent 230 both positioned on or substantially near the ceiling to deliver hot/cold air to the space and to discharge the “used air” out of the space. The air delivery system 200, like the prior art air conditioning system shown in FIG. 1, includes an air supply vent 212 and an air return vent 230 in each room.

As illustrated in FIG. 2A, the air flow in each room may be improved easily and inexpensively simply by attaching an air supplier 220 to the supply vent 212. In one or more embodiments, the air supplier 220 can include a pillar or columnar structure. The pillar can have any desired shape such as, rectangular, square, polygonal, or circular shape. The air supplier 220 or pillar can be directly attached to or positioned flush beneath an existing air supply vent 212 to modify the airflow within the enclosure without reworking or extensively modifying the ceiling, walls or any of the basic structure of the enclosure or an existing HVAC system that provides air supply to the enclosure. Generally, the air supplier 220 is equipped with one or more air supply vent openings 225 at or near the floor of the enclosure. Thus the air entering the room is blown in at or near the bottom of the enclosure to stir the air within the enclosure before exiting through the return vent.

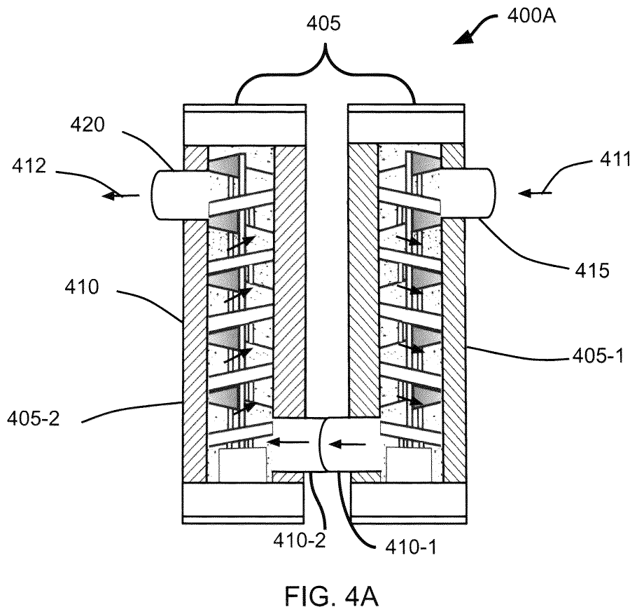

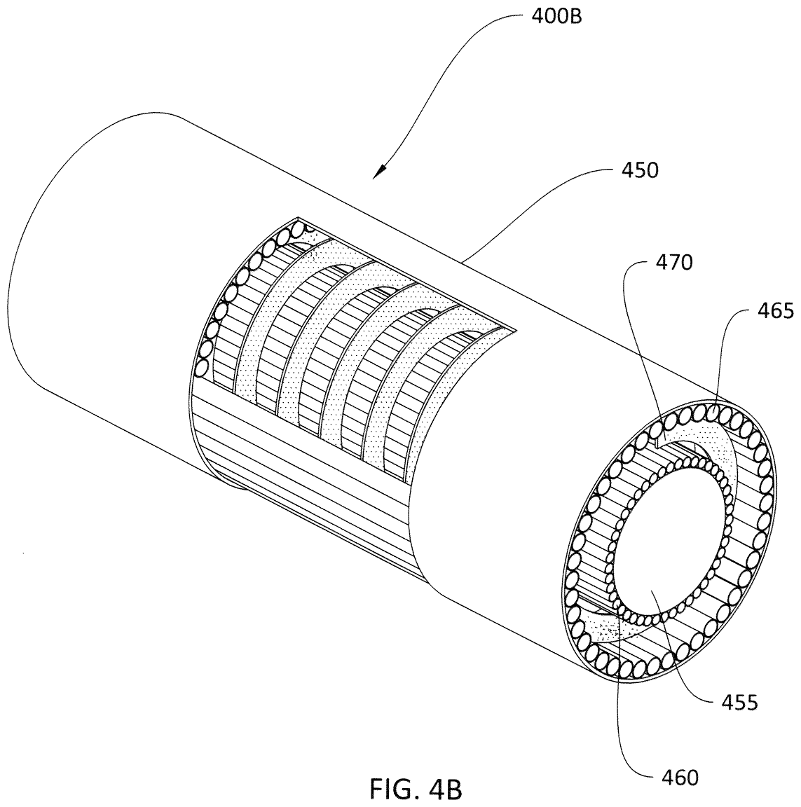

The system 200 may be simply modified to circulate disinfected air throughout the enclosure 201 by simply adding a disinfection unit 260 to the air supplier 220. For example, a disinfection unit disclosed in FIG. 4B can be embedded within the air supplier 220. This system not only improves air circulation within the enclosure but also provides a disinfection means to ensure that disinfected air is circulated throughout the enclosure, as well as make sure that the air leaving the room is disinfected.

The system 200 illustrated in FIG. 2C shows how the air supply vent 212 can be connected directly to a disinfection unit 260 that is then attached to more than one air distribution lines 215 that are in communication with a number of air suppliers 220, thereby providing incoming conditioned, disinfected air in different areas of the room. Generally, each of these additional air suppliers 220 is equipped with one or more air supply vent openings 225 at or near the floor of the enclosure.

In existing air conditioned multi-roomed structures such as a hospital, a nursing home, or an emergency clinic, each room is provided with an air supply vent 212 and an air return vent 230. Embodiments of the invention shown in FIGS. 2A, 2B, or 2C can thus be independently implemented in each room. Thus, some rooms may be designed to better circulate air conditioned air throughout the room, while other rooms may be designed to circulate disinfected, air conditioned air throughout the room. For example, a doctor's waiting room may circulate air conditioned air, while an examination room may be renovated to circulate disinfected air conditioned air.

Similarly, one or more return air lines can be attached to the return vent 230, thereby providing multiple return air lines in different areas of the enclosure. The additional air return lines can be independently added to an existing air return vent 230 to modify the airflow within structures or enclosures without reworking the ceiling, walls, or basic structure of the enclosures within the structure.

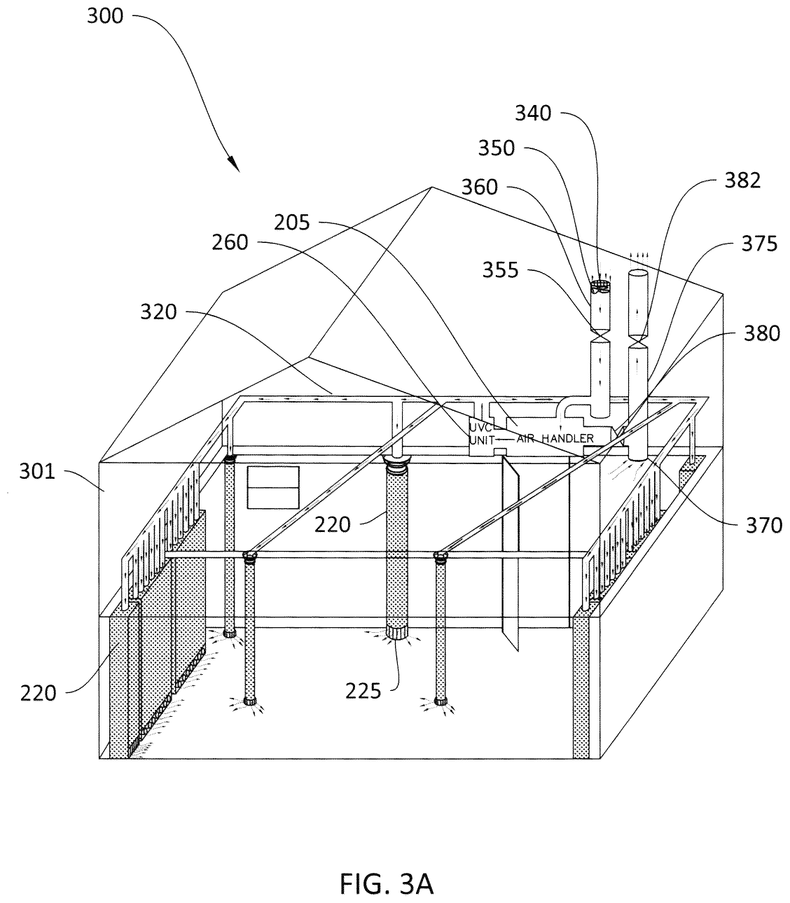

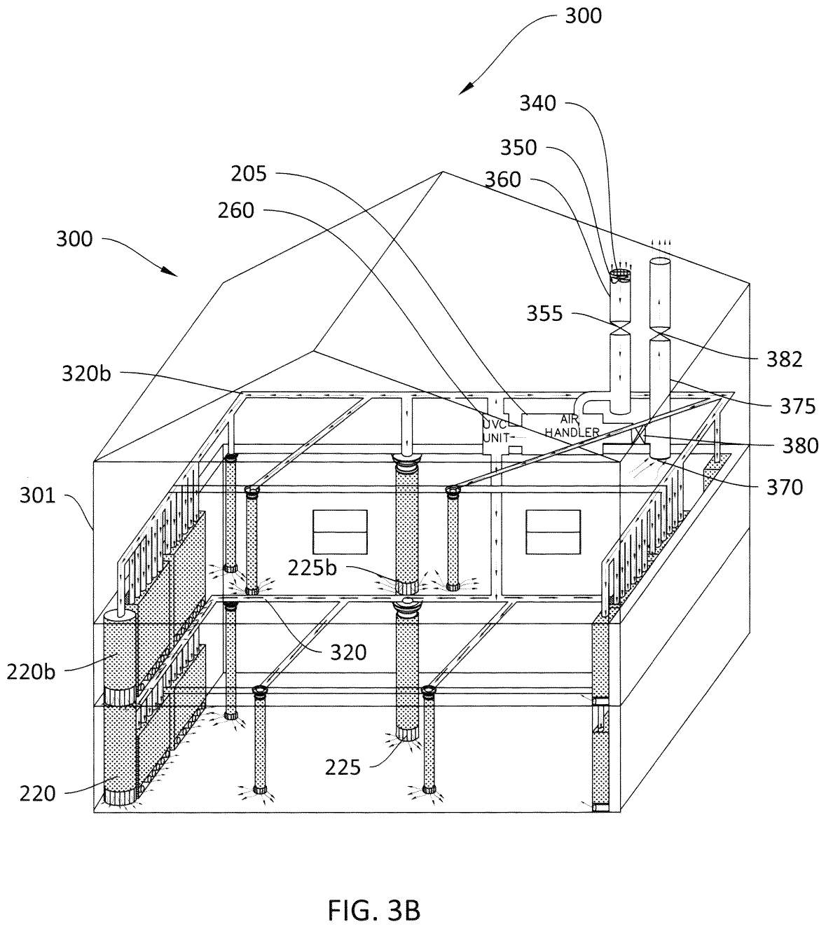

Another embodiment of the system 300 is shown in FIGS. 3A and 3B. FIG. 3A illustrates the system 300 implemented in a single enclosure 301, while FIG. 3B illustrates how the system 300 can be implemented in a multi-storied building. The system 300 comprises a fresh air intake pipe 360 for routing fresh air (shown as “arrows”) from the atmosphere/outside to an air handler 205 of a central air conditioning system. The fresh air intake pipe 360 can also include a screen 340 for stopping unwanted matter including, leaves, insects, birds, pollen, etc. from entering the air handler 205. The screen 340 can include a filter, such as, a HEPA air filter. One or more fans 350 can be provided proximal to the screen 340 to suck air into the fresh air intake pipe 360. The fresh air intake pipe 360 further includes a bi-directional valve 355 which can be toggled between an open and closed position.

Filtered ambient air flows through the fresh air intake pipe 360 to the air handler 205. The air handler routes the conditioned air through a disinfection unit 260 (exemplary embodiments of UVC disinfection units are shown in FIGS. 4A and 4B). The disinfected air from the UVC disinfection unit is then sent into the enclosure 301 through an air supply duct 320. The air supply duct may send the conditioned disinfected air directly into the enclosure through one or more air suppliers 220. As mentioned previously, the air suppliers can be pillars or columnar-structures. Alternatively, the air supply duct may send the conditioned disinfected air through the air supply vent 212. The disinfected air supply duct 320 can be coupled to one or more air suppliers 220 that are projected downward into the enclosure 201. The air suppliers 220 may be situated anywhere within and/or along the periphery of the enclosure as seen in FIG. 3A. As with the air supply system 200, the air suppliers do not have to be enclosed within the walls of the enclosure. The disinfected air supply is thus passed downward through the air suppliers and then out into the enclosure through the air supply vent openings 225 located at or near the base of the enclosure 301.

The air distribution system 300 described above and represented in FIG. 3A is the same as system as shown in FIG. 3B, except there are two air supply ducts, air supply duct 320 for the ground floor and 320b for the upper floor. The system for the upper floor is the same as for the ground floor with the air supply duct 320b sending conditioned disinfected air through the air suppliers and out through the air supply vents.

Advantageously, the supplied disinfected air has to travel the entire height of the enclosure 301 before it is routed through a return vent 370 located at or proximal to the ceiling. The return vent 370 is connected to the return duct 375. The return duct 375 is configured to route airflow from the enclosure 301 to either the air handler or the outside/atmosphere.

The return duct 375 can include a pair of bi-directional valves 380 and 382. Valve 380 is located on a branch of the return duct, proximal to the air handler 305. Valve 380 can be configured to be in a closed position to prevent used/returned air from flowing into the air handler 305; however, it can be opened when the valve 355 on the fresh air intake pipe 360 is closed. When valve 380 is closed, the returned air flows from the enclosure 301 out to the atmosphere through the return duct 375. The valve 382 is opened to allow the used air to be vented to the atmosphere. However, it is understood that when valve 382 is opened, valve 380 is closed to maintain air pressure in the enclosure 301.

Under normal circumstances, the valve 355 on the fresh air intake pipe 360 and the valve 382 on the return duct are closed, and the valve 380 on a branch of the return duct remains open. Such an arrangement facilitates the recirculation of disinfected conditioned air in the enclosed space; however, as described earlier, the supplied air is forced to flow from the ground or floor level to the return vent 370 on the ceiling. This process not only improves the cooling/heating of the enclosure 201, but it can also transport microorganisms/pathogens settled in the floor of the enclosure 301 through the return duct 370 to the air handler 305 and UVC unit 260 where it can be disinfected. The UVC light in the disinfection unit can destroy/kill the pathogens. The air in the lower half of the enclosure 201 is completely swept out by the treated disinfected air coming out from the air supply vent openings 225 and is constantly recirculated, resulting in a self-cleaning, scavenging airflow for air conditioners with incorporated UVC disinfection.

In one embodiment, the incoming airflow into the disinfection unit 260 is configured to be routed along a serpentine pathway within a housing of the disinfection chamber. The microorganisms in the airflow are exposed to the germicidal far UV-C light produced by the light sources for an optimal duration resulting in their neutralization. The disinfected air is discharged to the pillars and delivered/supplied through air supply vent openings 225 which are located at the floor level or substantially near the floor level in the room.

In certain circumstances, according to an embodiment, where an infected person is present in the enclosure 201, the valve 355 is opened for filtered ambient air to flow into the air handler 305 through the fresh air intake pipe 360. Also, the valve 380 remains closed and the valve 382 remains open for the used air to return to the atmosphere through the return duct 375. Thus, the system 300 ensures that the air supplied to the enclosure 301 through the alternate air supply duct 320 is substantially devoid of recirculated air and airborne microorganisms and is “totally clean” air conditioned or heated air.

In one or more embodiments, the columns or pillars can be embedded with or connected to tubes for inflow and outflow of air.

Exemplary air disinfection units (for use in the various embodiments of the air delivery system described herein) are disclosed in U.S. Ser. No. 18/328,463, the content of which is incorporated herein by reference in its entirety. An exemplary embodiment of an air disinfection unit 400A is illustrated in FIG. 4A. The air disinfection unit 400A has one or more modules 405-1, 405-2 (“405”). A first module 405-1 includes a housing having an air inlet 415 and a housing outlet 410-1. A second module 405-2 is connected in series to the first module 405-1. Housing outlet 410-1 is connected to housing outlet 410-2 of the second module 405-2. The connection may be in the form of bellows, so that the air from one module does not go out into the ambient air but is forced to go into the next module. The air inlet 415 allows ambient/incoming air 411 to enter the air disinfection unit 400A and the disinfected air 412 exits out the air outlet 420. The disinfection unit 400A can be expanded by increasing the number of UV disinfection units/modules 405. The disinfection unit 400A can include a plurality of ultraviolet lights.

In another embodiment, disinfection unit 400B can be used to disinfect air-conditioned airflow. As shown, the disinfection unit 400B includes a substantially cylindrical outer housing 450. The disinfection unit 400B further includes a cylindrical inner housing 455 that is positioned centrally within a cavity formed within the outer housing 450. The inner cylinder 455—which may be a solid/rod-like structure—extends the length of the outer housing 450. A plurality of UV light sources 470 is affixed around an outer surface of the inner cylinder while a plurality of UV lights 465 is affixed to an inner surface of the outer housing. The UV light sources 460, 465 are configured to emit germicidal far UV-C light. An airflow divertor 470 is positioned in the space between the UV light sources 460 and 465. The airflow diverter 470 is substantially spiral or helical. The airflow diverter 470 is configured to create a serpentine airflow pathway for the incoming (untreated) airflow such that any microorganisms in the air are exposed to the far UV-C lights for a substantially long period. The length and diameter of the housing 450 and the distance between each spiral turn of the airflow diverter 470 can be increased or decreased to increase or slow the passage of the airflow to be disinfected. The disinfection units, as shown in FIGS. 4A and 4B, can be incorporated within an existing air-conditioning system.

In one or more embodiments, one or more adapters or connectors can be used to couple the air suppliers (or columns/pillars) to the air vents or ducts.

It is understood that the enclosure can have multiple ducts and vents, and all such embodiments are withing the scope of the present invention. Furthermore, the components of conventional air conditioning systems (such as, air handlers/evaporators/blowers/furnaces, condensers, etc.) are well known in the art and have not been reiterated herein.

The many features and advantages of the invention are apparent from the written description and, thus, to cover all such features and advantages of the invention. Accordingly, all suitable modifications and equivalents may be resorted to as falling within the scope of the invention.

I claim:

1. An air flow delivery system for delivering conditioned air to an enclosure, comprising:
(a) an air handler coupled at a first end to an air supply duct and at second end to an air return duct;
(b) an air supply vent attached to the air supply duct, wherein the air supply vent is in or near the ceiling of the enclosure;
(c) an air return vent attached to the air return duct, wherein the air return vent is in or near the ceiling of the enclosure; and
(d) an air supplier connected at a first end to the air supply vent, wherein the air supplier projects downward from the air supply vent into the enclosure near a floor of the enclosure, wherein an opening in the air supplier providing an air passage from the interior of the air supplier to the enclosure.

2. The system as claimed in claim 1, wherein multiple downwardly projecting air suppliers are provided within the enclosure.

3. The system as claimed in claim 1, wherein the air supplier is a pillar or column.

4. The system as claimed in claim 1, wherein the air supplier includes a disinfection unit therein.

5. The system as claimed in claim 1, wherein the air supplier opening is near a bottom end of the air supplier.

6. An air flow delivery system for delivering conditioned air to an enclosure, comprising:
(a) an air intake pipe having a bidirectional intake valve therein, wherein when the bidirectional intake valve is in an open state, ambient air enters the air flow delivery system, and wherein when the bidirectional intake valve is in a closed state, ambient air is prevented from entering the air flow delivery system;
(b) an air return duct having a first and a second bidirectional valve therein, wherein when the first bidirectional valve is in an open state and the second bidirectional valve is in a closed state ambient air enters the air flow delivery system, and wherein when the first bidirectional valve is in a closed state and the second bidirectional valve is in an open state, ambient air is prevented from entering the air flow delivery system;
(c) an air handler coupled to the air intake pipe below the bidirectional intake valve in the air intake pipe, the air supply duct, and the air return duct;
(d) an air supply duct or air supplier connected to a second end of the air handler and extending into an interior of the enclosure, wherein the air supply duct delivers conditioned air from the air handler to one or more air supply vents positioned within the enclosure, wherein the air supply duct extends vertically from a ceiling of the enclosure toward a floor of the enclosure; and
(e) one or more air return vents connected to a return duct for routing used air from the enclosure.

7. The system as claimed in claim 6, wherein a disinfection unit is in communication with the air handler and the air supply duct.

8. The system as claimed in claim 6, wherein the air intake pipe includes a filter therein.

9. The system as claimed in claim 6, wherein the air intake pipe includes a fan therein.

10. The system as claimed in claim 6, wherein the one or more air supply vents are arranged along a perimeter of the enclosure.

11. The system as claimed in claim 6, wherein the one or more return vents are located at or proximal to a ceiling of the enclosure.

12. The system as claimed in claim 6, wherein the return duct is connected to the one or more return vents.

13. The system as claimed in claim 7, wherein the disinfection unit is configured to emit ultraviolet-C germicidal light.
